(19) United States
(12) Patent Application Publication
(10) Pub. No.: US 2014/0155272 A1
(43) Pub. Date: Jun. 5, 2014

(54) Use Of Diatomaceous Earth As The Base Substrate For Nucleic Acid Tags

(71) Applicant: SRC, INC., North Syracuse, NY (US)

(72) Inventors: Laura T. Bortolin, Westford, MA (US); Mary F. Swartz, Princeton, NJ (US); Garrett D. Liddil, Fayetteville, NY (US); Adam J. Lowe, Syracuse, NY (US)

(73) Assignee: SRC, INC., North Syracuse, NY (US)

(21) Appl. No.: 13/693,425

(22) Filed: Dec. 4, 2012

(51) Int. Cl. C12Q 1/68

(52) U.S. Cl. CPC C12Q 1/6834; Y10S 977/773
USPC 506/2; 506/16; 435/6.12; 506/9; 435/6.11; 977/773; 977/902

(57) ABSTRACT

A nucleic acid tag comprising: a nanoparticle nucleotide-support platform attached to a plurality of nucleic acid molecules, each of said nucleic acid molecules comprising identifying information, with a spacer located between the nanoparticle nucleotide-support platform and the identifying information, and where the nanoparticle nucleotide-support platform comprises diatomaceous earth; and an encapsulant surrounding the nanoparticle nucleotide-support platform.

### BACKGROUND OF THE INVENTION

1. Field of the Invention

The present invention relates to systems and methods using nucleic acid tags, and, more particularly, to the design of nucleic acid tags using diatomaceous earth.

2. Description of the Related Art

The physical characteristics of a nucleic acid molecule make it uniquely suitable for use as a secure information-storage unit. In addition to being odorless and invisible to the naked eye, a nucleic acid molecule can store vast amounts of information. It has been estimated that a single gram of deoxyribonucleic acid (“DNA”) can store as much information as approximately one trillion compact discs (“Computing With DNA” by L. M. Adleman, Scientific American, August 1998, pg 34-41).

Nucleic acid molecules are also resilient to decay, even in vitro. Although a nucleic acid molecule typically begins to breakdown when exposed to chemicals, radiation, or enzymes, some nucleic acid molecules can survive for thousands of years. For example, scientists have sequenced the Neanderthal genome using DNA molecules that were recovered from remains dating at least 38,000 years old.

Additionally, nucleic acid molecules are both ubiquitous in nature and largely uncharacterized, with only a fraction of the world's organisms having been sequenced. As a result of this uncharacterized environmental background noise, inadvertent detection of a man-made nucleic acid molecule is unlikely.

To employ the many beneficial characteristics of nucleic acids, these molecules can be incorporated into a secure tag. These tags can be composed of deoxyribonucleotides, ribonucleotides, or similar molecules composed of nucleic acids that are either artificial (such as nucleotide analogues) or are otherwise found in nature. The nucleic acids can range from very short oligonucleotides to complete genomes.

Once a nucleic acid tag is created it can be used for numerous unique security applications including to: (i) detect illicit tampering with physical objects; (ii) secure the privacy of a room or building; (iii) send encoded messages between individuals; (iv) detect a tagged individual or object at a distance; (v) track the recent travel history of an individual or object; or (vi) monitor a location of interest, among many other uses.

There is, however, a continued demand for new and efficient mechanisms for producing more robust nucleic acid tags both efficiently and economically.

### BRIEF SUMMARY OF THE INVENTION

It is therefore a principal object and advantage of the present invention to provide an economical and efficient nucleic acid tag design comprising diatomaceous earth.

Other objects and advantages of the present invention will in part be obvious, and in part appear hereinafter.

In one application, a nucleic acid tag comprises a nanoparticle nucleotide-support platform attached to a plurality of nucleic acid molecules, each of the nucleic acid molecules comprising identifying information, wherein a spacer is located between the nanoparticle nucleotide-support platform and the identifying information, and further wherein the nanoparticle nucleotide-support platform comprises diatomaceous earth; andan encapsulant surrounding the nanoparticle nucleotide-support platform.

In one aspect, the encapsulant is adapted to prevent degradation of the plurality of nucleic acid molecules.

In one aspect, each of the plurality of nucleic acid molecules is composed of nucleotides selected from the group consisting of ribonucleotides, deoxyribonucleotides, and nucleotide analogues.

In one aspect, each of the plurality of nucleic acid molecules is an oligonucleotide.

In one aspect, each of the plurality of nucleic acid molecules is genomic deoxyribonucleic acid ranging from two nucleotides to the entire genome.

In one aspect, information is encrypted within the genomic deoxyribonucleic acid molecule by altering the sequence of nucleotides.

In one aspect, the nucleic acid tag comprises a retroreflector.

In one aspect, the nucleic acid tag comprises a luminescent compound.

In another application, a method for determining whether an item has moved through a geographic location comprises: (i) creating a nucleic acid tag comprising a nanoparticle nucleotide-support platform attached to a plurality of nucleic acid molecules, each of the nucleic acid molecules comprising identifying information, wherein a spacer is located between the nanoparticle nucleotide-support platform and the identifying information, and further wherein the nanoparticle nucleotide-support platform comprises diatomaceous earth; (ii) seeding the geographic location with the nucleic acid tag; and (iii) examining the item for the presence of the nucleic acid tag.

In one aspect, the nucleic acid tag is analyzed by sequencing all or part of the nucleic acid molecule.

In one aspect, each geographic location is seeded with a unique nucleic acid tag.

In yet another application, a method for backtracking the travel history of an item comprises: (i) creating two or more nucleic acid tags, each tag comprising: a nanoparticle nucleotide-support platform attached to a plurality of nucleic acid molecules, each of the nucleic acid molecules comprising identifying information, wherein a spacer is located between the nanoparticle nucleotide-support platform and the identifying information, and further wherein the nanoparticle nucleotide-support platform comprises diatomaceous earth; (ii) seeding each of two or more geographic locations with said nucleic acid tags, wherein each geographic location is seeded with a unique nucleic acid; (iii) examining said item for the presence of one or more nucleic acid tags; and (iv) identifying the geographic location associated with each nucleic acid tag detected on the item.

In one aspect, the method further comprises the step of extrapolating the point of origin.

In another application, a method for detecting a seeded nucleic acid tag in or on an item of interest comprises: (i) obtaining a nucleic acid tag, wherein the nucleic acid tag comprises a nanoparticle nucleotide-support platform attached to a plurality of nucleic acid molecules, each of the nucleic acid molecules comprising identifying information, wherein a spacer is located between the nanoparticle nucleotide-support platform and the identifying information, and further wherein the nanoparticle nucleotide-support platform comprises diatomaceous earth; (ii) adding the nucleic acid tag to the item of interest; (iii) sampling a portion of the item of interest for the presence of the nucleic acid tag; and (iv) detecting the presence of the nucleic acid tag in the sample.

In one aspect, the presence of the tag on an exterior surface indicates tampering.

In one aspect, the presence of the nucleic acid tag authenticates the item of interest.

In one aspect, the step of adding the nucleic acid tag to the item of interest comprises incorporating the nucleic acid tag within a label or a package of the item of interest.

In one aspect, the step of adding the nucleic acid tag to the item of interest comprises incorporating the nucleic acid tag into a precursor of the item of interest.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a flowchart of an exemplary process for creating a nucleic acid tag in accordance with an embodiment;

FIG. 2 is a flowchart of an exemplary process for using a nucleic acid tag in accordance with an embodiment;

FIG. 3 is a side view of a nucleic acid tag complex in accordance with an embodiment;

FIG. 4 is a side view of encapsulated nucleotide-derivatized nanoparticles in accordance with an embodiment;

FIG. 5 is a side view of an encapsulated nucleotide tag complex with marker elements incorporated into the encapsulant layer in accordance with an embodiment;

FIG. 6 is a side view of an encapsulated nucleotide tag complex with marker elements incorporated into the nanoparticles in accordance with an embodiment;

FIG. 7 is a side view of an encapsulated nucleotide tag complex with marker elements coating the outer surface of the encapsulant in accordance with an embodiment;

FIG. 8 is side view of an encapsulated nucleotide tag complex with marker elements coating the outer surface of the encapsulant in accordance with an embodiment;

FIG. 9 is a side view of an encapsulated nucleotide tag complex with marker elements trapped inside the tag by the encapsulant layer in accordance with an embodiment;

FIG. 10 is a flowchart of an exemplary process for using nucleic acid to detect tampering in accordance with an embodiment;

FIG. 11 is a flowchart of an exemplary process for using nucleic acid for authentication in accordance with an embodiment; and

FIG. 12 is a flowchart of an exemplary process for using nucleic acid in accordance with an embodiment.

### DETAILED DESCRIPTION OF THE INVENTION

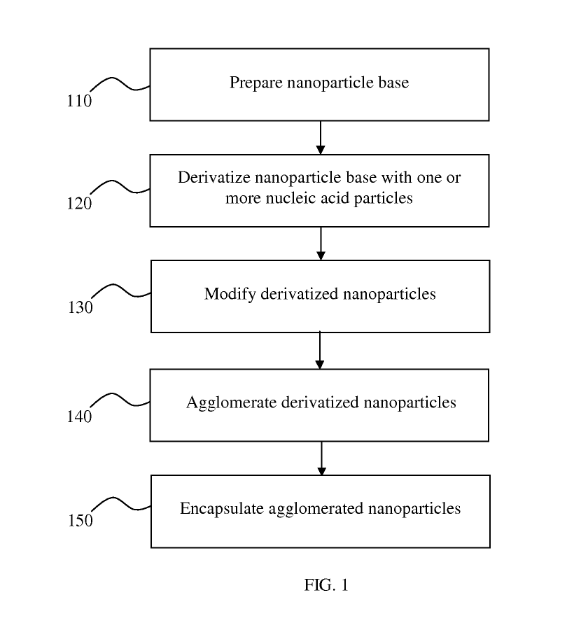

Referring now to the drawings wherein like reference numerals designate identical or corresponding parts throughout the several views, there is shown in FIG. 1 a flowchart of an exemplary process for creating a suitable nucleic acid tag in accordance with an embodiment of the present invention. As an initial step 110, a nanometer-sized particle (“nanoparticle”) platform is prepared for attachment to the nucleic acid molecule(s). A platform is used to make the nucleic acid more accessible to downstream analysis and prevent nucleic acid loss if any portion of the encapsulating layer is compromised.

The platform is any compound that can be attached to nucleic acid without unintentionally degrading or altering the nucleic acid sequence. For example, the platform can be a lightweight, durable, non-water soluble, and chemically inert structure composed of silica or polystyrene. Additionally, the nanoparticle platform should be composed of a compound that does not inhibit any downstream analysis of the nucleic acid molecules, including tag detection and polymerase chain reaction (“PCR”).

In one application, the platform comprises diatoms, including but not limited to diatomaceous earth. Diatomaceous earth, also known as diatomite, is a naturally occurring siliceous sedimentary rock that consists of fossilized remains of diatoms, a type of unicellular algae. Diatomaceous earth has several existing applications, including as an abrasive, for filtration, and as an absorbent.

The diatomaceous earth used for the nanoparticle component of the nucleic acid tags can be formed by pulverizing or otherwise reducing the size of larger particles, including mined rocks or blocks, or particles of diatomaceous earth can be purchased from a supplier at or near the desired size. Although diatomaceous earth is comprised of particles that range from under 1 micrometer to more than 1 millimeter, a specific size or size range can be selected by sieving, filtering, or by purchasing the desired size or species of diatoms.

Once the particles of the diatomaceous earth are the proper size, they can be derivatized with the nucleic acid using any of the methods or approaches described or suggested herein. One benefit of diatomaceous earth is that it does not inhibit the downstream PCR reactions that might be needed to identify and/or characterize the tag or the nucleic acid component of the tag.

At step 120, the nucleic acid molecule is attached to the prepared nanoparticle platform. The nucleic acid can be any natural or artificial nucleic acid, including but not limited to deoxyribonucleotides, ribonucleotides, oligonucleotides, nucleic acid analogs, and similar molecules that are either artificial or are otherwise found in nature, as well as combinations of any or all of the above. The nucleic acids can range from a very short sequence to a complete genome, for example. The nucleic acid molecules are optimally attached to the nanoparticle to facilitate later analysis. In a preferred embodiment, a chemical linker is used to connect the nucleic acid to the nanoparticle platform. This chemical linker must keep the nucleic acid securely tethered to the nanoparticle while avoiding inhibition of the detection or analysis of the tag and nucleic acid. Although the chemical linker can be chosen to provide a permanent covalent link between the nucleic acid and the nanoparticle platform, it could also be a compound that quickly and efficiently releases the nucleic acid at a certain temperature or after exposure to a release compound.

The nucleic acid molecule can also be designed to promote analysis. For example, to avoid steric hindrance or unwanted intermolecular interactions, the molecule can include nucleotide spacers between the chemical linker or nanoparticle base and the information-coding segment of the nucleotide sequence. Spacing between 1 and 100 bases has been optimal for current applications, although this may vary as new applications are considered.

The concentration of nucleic acid molecules on the nanoparticle platform is also an important factor in downstream analysis. If the molecules are too concentrated, steric hindrance prevents the primer and polymerase from efficiently binding the proper segments of the nucleic acid molecules. If the molecules are too sparse, the PCR signal will be diminished and can result in false negatives. In a preferred embodiment, a concentration of about 1×10⁸ to 1×10¹² nucleic acid molecules per square centimeter is the optimal concentration for robust PCR signal.

According to another embodiment, the nucleic acid molecules are attached to the nanoparticle platform by synthesizing the nucleic acid directly onto the nanoparticle platform. There are a variety of methods for performing this step, but according to a preferred embodiment the platform—such as a diatom—is silanated with an alcohol-terminated silane, and then the functionalized diatom is used as a solid support for oligonucleotide synthesis. As an optional method, the silate could be treated with a base to deprotonate the silanols on the surface, washed and dried in solvent, and then used as the solid support for oligonucleotide synthesis.

At step 130, which can occur at the position shown in the flowchart or before or after any other step after derivatization of the nanoparticles, the derivatized nanoparticles can optionally be modified for any purpose, use, or design. For example, a flame or fire retardant can be added to the derivatized nanoparticles. The flame or fire retardant is preferably anything known by those skilled in the art to inhibit combustion or reduce the temperature of associated material in response to high temperatures, including but not limited to Nomex®, GORE-TEX®, Kevlar®, aluminum hydroxide, magnesium hydroxide, hydromagnesite, calcium silicate, or halocarbons, among many others. While some compounds provide the tag with resistance to combustion, others provide the tag with thermal protection by absorbing heat in an endothermic reaction, through chemical degradation, or by otherwise protecting the tag from high temperatures.

The derivatized nanoparticles can also be modified to include an odorant. The odorant can be anything known to be capable of detection by mechanical means or by human or animal means (i.e., olfaction detection). The odorant can comprise anything known by those skilled in the art to be capable of detection, including a single aromatic, a blend of aromatics, or a commercially available synthetic chemical, among many others. Since the surfaces on which the odorant might be detected will vary, the odorant will preferably be unique or distinctive enough to be detected over random odorants present on these surfaces or in the surrounding environment. Although according to one embodiment the odorant is capable of detection by humans and/or animals, in the preferred embodiment the odorant can only be detected by animals and/or electronic means, thereby evading human detection. For example, mechanical means such as an “electronic nose” could be programmed or trained to recognize the odorant and alert the user to its presence. In a preferred embodiment, the sensor provides quantitative information about detection and is sensitive enough to detect very minute or trace amounts of the odorant.

Lastly, the tag can also be modified with other compounds to provide additional desired characteristics including but not limited to color, luminescence, or protection against ultraviolet radiation.

At step 140 of the exemplary method, the nucleic acid-derivatized nanoparticles are agglomerated. Agglomeration protects the nucleic acid molecules from degradation and facilitates encapsulation. To agglomerate the particles to the desired size range, the nanoparticles are vacuum dried, milled, and sieved.

Compounds might be used or incorporated into the tag to promote disagglomeration of the agglomerates prior to PCR analysis. These compounds might be bovine serum albumin, salmon sperm DNA, carbohydrates, polyvinyl alcohol, fructose, or chitosan, among others. With more nucleic acid exposed during dissolution, subsequent analysis will be faster and more sensitive.

After the nanoparticles are agglomerated, the agglomerates are encapsulated at step 150. The encapsulant protects the nucleic acid from degradation by ultraviolet light, hydrolysis, enzymatic digestions, chemical degradation, or any other means. Additionally, the encapsulant can be designed such that it does not hinder analysis of the nucleic acid molecules. For example, the encapsulant should not contain any compounds that would inhibit or prevent a PCR reaction, although efficient removal of the encapsulant before PCR analysis would eliminate this requirement. Additionally, the encapsulant should enhance the ability of the tag to discretely attach to people and objects. If covertness is required, the encapsulant can be designed to deter detection.

The encapsulating layer can also be designed with surface moieties added to the inner or outer surfaces of the encapsulant or incorporated into the encapsulant material. The moieties are designed to facilitate a particular use of the nucleic acid tag. For example, the moiety can be hydrophobic to enable stickiness or contain antibodies designed for specific targeting. The molecular interactions between the moiety and a target compound can range from simple electrostatic interactions to antibody-antigen recognition. The moiety can also promote detection of the nucleic acid tag.

To protect the nucleic acid from degradation, the encapsulating layer can be coated with or include another functional layer of material. For example, the encapsulant can be coated with or include a non-water-soluble compound to prevent access to water or similar molecules. The encapsulant can also be coated with or include a UV-blocking compound such as titanium dioxide to prevent UV-induced degradation of the nucleic acid molecules.

In yet another embodiment, the nucleic acid tag comprises just nucleic acid, or nucleic acid in combination with a structure or base other than a nanoparticle. For example, the nucleic acid may be unencumbered, or it may be tethered (covalently or non-covalently) to a structure or base. There may be many copies of the nucleic acid, or just a few copies, and can range from a very short sequence to a complete genome, for example. The nucleic acid can be connected to the structure or base by a chemical linker. Although the chemical linker can be chosen to provide a permanent covalent link between the nucleic acid and the structure or base it could also be a compound that quickly and efficiently releases the nucleic acid at a certain temperature or after exposure to a release compound. The nucleic acid molecule can also include nucleotide spacers between the chemical linker or nanoparticle base and the information-coding segment of the nucleotide sequence in order to avoid steric hindrance or unwanted intermolecular interactions. Spacing between 1 and 100 bases has been optimal for current applications, although this may vary as new applications are considered.

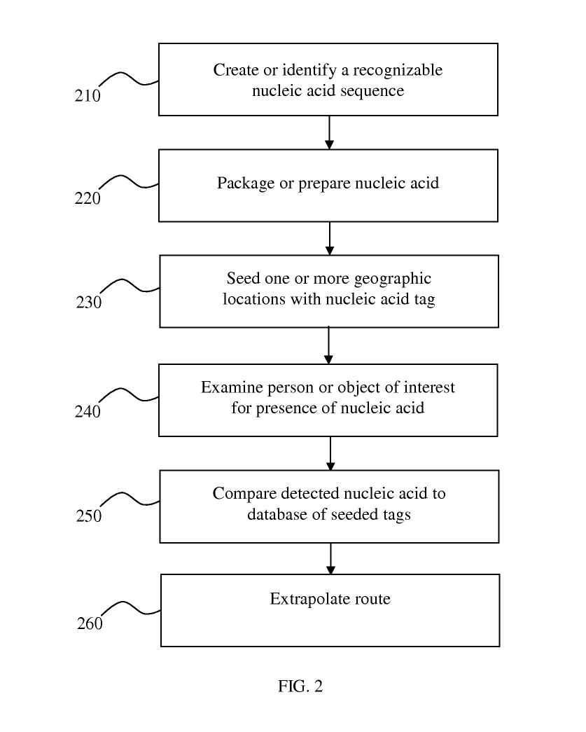

FIG. 2 is a schematic representation of an embodiment of a security method according to the present invention. More specifically, the figure represents characterization of the recent travel history of point of an item. An item can be any person or object of interest. Seeding an area with tags that naturally or artificially adhere to objects (including people or animals) provides a mechanism for identifying the origin of those objects simply by analyzing the adhering tags. Similarly, by seeding different areas with discernibly different tags it is possible to backtrack the geographic path that an object has followed. Such a mechanism would allow the seeder—the person or organization who seeded and will analyze the tags—to identify the recent travel history of the person or object; to quickly identify people or objects that have traveled through seeded areas; and to identify vehicles that have traveled through seeded areas and might carry dangerous cargo such as explosives, among other uses.

As an initial step 210, a suitable nucleic acid sequence is characterized or created. In one embodiment of the present invention, the sequence ranges from a short oligonucleotide to an entire genome and is generated through any of the various known methods of natural or artificial nucleic acid synthesis. The nucleic acid can be completely composed of either natural nucleic acids which normally compose the genomes of organisms, artificial nucleic acids, or any combination thereof.

In a preferred embodiment, the nucleic acid molecules contain primer-binding sequences surrounding unique nucleotide sequences. The unique nucleotide sequence contained between the primers can encode information that corresponds to an identification, location, date, time, or other data specific to that unique sequence. Since analysis of every nucleic acid molecule can use the same primers, the analysis can be performed faster and more efficiently.

The primer sequences, whether they are unique or identical for each location or use, are chosen to avoid cross-reactions with naturally-occurring nucleic acid molecules in the environment in which the nucleic acid is located. Although only a fraction of natural nucleic acid molecules on Earth have been characterized by scientists, the search of nucleic acid repository databases such as GenBank®, the National Institutes of Health database containing all publicly available DNA sequences, could be a preliminary step in constructing the primer sequences.

In one embodiment of the current invention, unique groupings of nucleotides are assigned a specific letter, number, or symbol value in order to encode information within the sequence. By placing the unique groupings in order, information can be encrypted into the nucleotide sequence. To further increase the security of the information, advanced encryption algorithms can be used to assign letter, number, or symbol values to specific nucleotides or nucleotide groupings. Additionally, the encryption system can be periodically changed to prevent decryption by intercepting entities.

The nucleic acid can also be encoded to contain information other than a string of letters, numbers, and symbols. For instance, the sequence can be a random sequence that corresponds to the item, location, or date that the object of interest will be seeded. Alternatively, the tag can be as simple as a single nucleic acid change in a previously identified or known sequence. For example, the nucleotide sequence can be embedded in a full or partial genomic sequence corresponding to an organism which naturally exists in the location to be seeded. Modifications to the natural nucleic acid sequence, known only to the creator of the tag, can be made such that the changes resemble natural variations of the sequence and thus fail to arouse suspicion, even by individuals that might suspect such tags are present.

To decrypt the encoded information according to this system, an individual will need: (1) knowledge that encoded nucleic acid is present; (2) knowledge of the specific location of the information within the nucleic acid in order to use the appropriate primers for amplification and sequencing reactions; (3) access to a PCR machine and reagents; and (4) the encryption algorithm, or, alternatively, complex decryption capabilities.

Although creating the nucleic acid target within the genome of an naturally-occurring organism provides numerous benefits, both in vivo and in vitro DNA replication occasionally introduces random errors into a DNA sequence despite the actions of proof-reading and repair enzymes. By deleting one or more nucleotides or frame-shifting the nucleic acid sequence, these mutations can disrupt any encrypted information contained therein. Computer algorithms are used to restore the information by recognizing and repairing the errors. For example, if a mutation adds one or more nucleotides to a pre-defined sequence and disrupts the information, the algorithm removes single or multiple nucleotides from the sequence until the information is corrected. Similarly, if a mutation removes one or more nucleotides, the algorithm systematically adds nucleotides to the sequence until the information is corrected. The algorithm must also be robust enough to decrypt sequences that contain more than one type of error-inducing mutation, and must be capable of recognizing when the information contained with the nucleic acid has been restored.

In step 220 of the exemplary method shown in FIG. 2, the nucleic acid is packaged, prepared, or otherwise modified prior to use. Preparation of the nucleic acid can range from little or no preparation or modification to an extensive series of steps for modifying the nucleic acid. For example, the nucleic acid can be used to derivatize nanoparticles, as described above, or can be added to another structure or base.

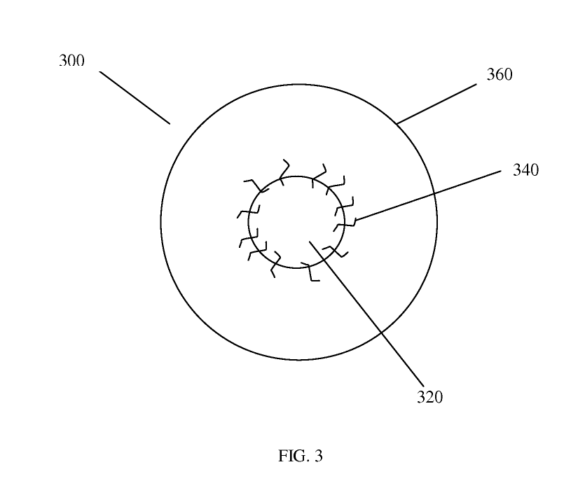

As another example, the nucleic acid can be packaged into an appropriate tag complex. To avoid potentially harmful environmental side-effects, the tag can be large enough to avoid being inhaled by people or organisms but small enough to be covert. FIG. 3 represents one embodiment of this tag structure. Tag 300 is composed of a single nucleotide-support platform 320, nucleic acid 340, and encapsulant 360.

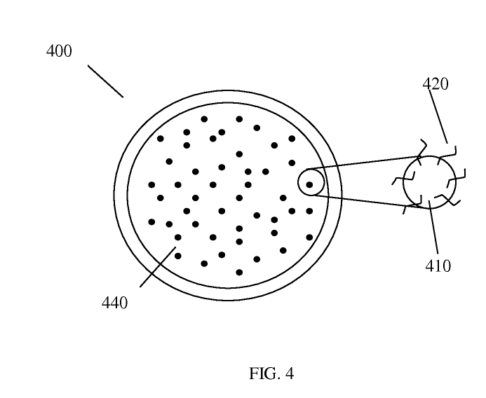

FIG. 4 is a side view of another embodiment of a tag structure. Tag 400 is composed of nucleotide-support platform 410 derivatized with nucleic acid 420 and surrounded by encapsulant 440. Similar to the tag in FIG. 3, tag 400 contains nucleic acids that are contained within an encapsulant that protects the sequence without inhibiting later analysis. Unlike the bead platform used by the tag in FIG. 3, nucleotide-support platform 410 is composed of nanoparticles. Tag 400 can contain thousands, millions, or even billions of nucleotide-derivatized nanoparticles within the encapsulant layer.

In yet another embodiment, the tag complex can be modified to include, comprise, or be associated with an additional element 500 such as a unique identifier, a fire or flame retardant, a UV-protectant, a waterproof element, and/or an odorant, among many other types of modification. For example, a fire or flame retardant can protect the tag by resisting combustion or lowering high external temperatures. A fire- or high temperature-resistant tag can be used for many different applications, including those where the tag is expected to be exposed to fire or the high temperature of an explosion. The tags can be used to detect tampering in areas or on items or individuals suspected to be involved in the constructions of bombs or other incendiary devices, and the fire- or heat-resistant element would help the tamper tag survive the explosion, which could then be analyzed using downstream processes.

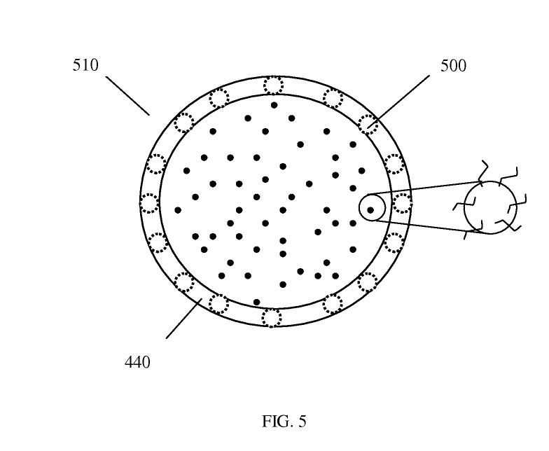

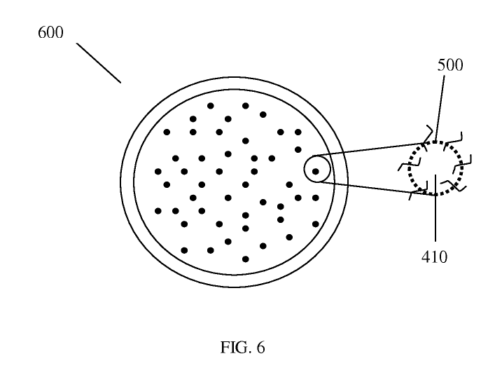

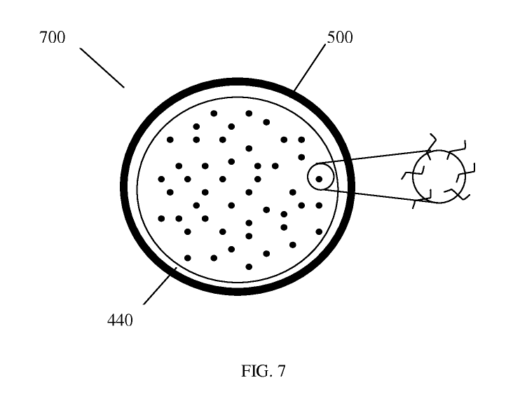

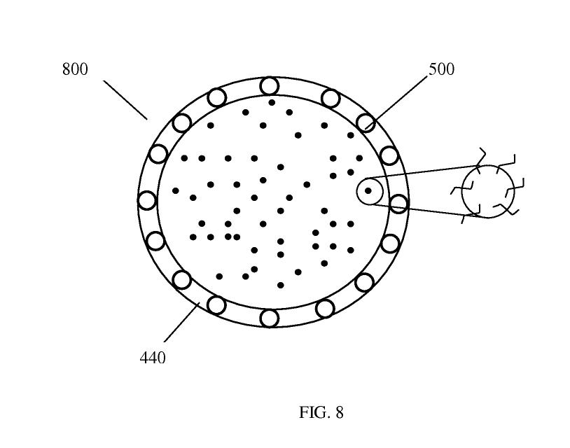

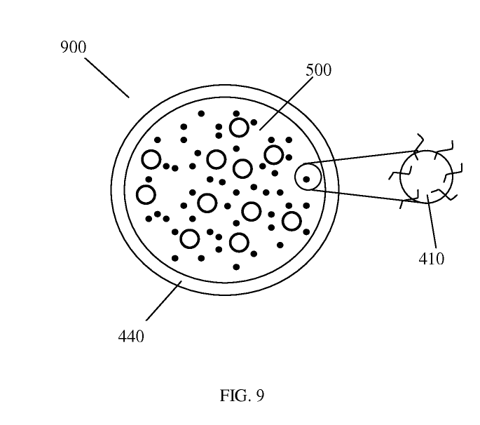

Additional element 500 can be incorporated into the tag in a number of different ways. For example, in FIG. 5 additional element 500 is incorporated into encapsulant 440 around tag 510. In FIG. 6, additional element 500 forms a portion of the structure or base 410 that the nucleic acid is bound to. In FIG. 7, additional element 500 forms a layer on the exterior surface of encapsulant 440. In FIG. 8, additional element 500 is incorporated into the exterior layer of tag 440. In FIG. 9, additional element 500 is separate from nucleotide-support platform 410 and encapsulant 440 but is trapped within the interior of tag 900.

While the embodiments depicted in FIGS. 5-8 are shown with nucleic acid derivatizing a nanoparticle, the nucleic acid may be unencumbered, or may be attached or in communication with another form of structure or base. None of these embodiments are meant to limit the potential scope of the invention, or fully describe the possible combinations of nucleic acid, support platform, and additional elements.

At step 230 of the exemplary method depicted in FIG. 2, one or more geographic locations are seeded with the tags. The locations are seeded with tags using any mechanism that will adequately disperse the tags at the desired concentration. For example, the tags can be seeded on and along roadways or paths using an automobile that has been modified to disperse the tags. The tags can also be discretely dispersed from the air using an airplane or remotely-controlled flying apparatus. Tags can even be seeded by individuals using hand-held dispersal systems.

To efficiently backtrack the movements of a person, vehicle, or object, each road within a given location can be seeded with a unique tag. As the vehicle moves through the location it picks up tags from each road it traverses. This system can be scaled up or scaled down to suit the needs of the seeder. For example, rather than seeding individual roads the seeder can use the tags to label large regions of land to backtrack large-scale movements. Alternatively, the seeder can scale down the method by seeding individual homes or buildings to identify individuals or objects that have entered those buildings.

In step 240 of FIG. 2, an item is examined for the presence of seeded tags. Once an object of interest is identified, the object can be examined for seeded tags using any mechanism designed to pick up tags from the surfaces of the object. For example, the tires, wheel wells, or underside of a vehicle can be swabbed for tags. If the object of interest is a person, the individual's clothes, shoes, hair, or skin can be swabbed for tags. If the object of interest is a post-blast fragment of an explosive device, the surfaces of the fragment can be swabbed for any tags that survived the explosion.

If the seeded nucleic acid contains, comprises, or was distributed in connection with retroreflectors, electromagnetic waves can be used to detect the presence of seeded nucleic acid. Scanning equipment shines light on the object of interest and looks for a wave front that is reflected along a vector that is parallel to but opposite in direction from the wave's source. This suggests that retroreflective tags are present on the exterior of the object and alerts the authorities that further investigation is necessary. This rapid and cost-effective identification of retroreflective tags is especially useful for high-throughput locations such as checkpoints and border crossings. Once the retroreflective tags are detected, they can be removed from the surfaces of the object for analysis of the attached nucleic acids to identify geographic locations.

The nucleic acid can also contain, comprise, or be seeded in connection with luminescent compounds that reveal their presence from a distance. Although the preferred embodiment uses fluorescent or phosphorescent photoluminescence, other embodiments may include chemiluminesent, radioluminescent, or thermoluminescent compounds. The photoluminescent compound is chosen such that absorption of a photon with a certain wavelength by the compound causes the emission of a photon with a different wavelength. The difference between the wavelength of the absorbed photon and the wavelength of the emitted photon depends on the inherent physical properties of the chosen compound.

In the preferred embodiment, the luminescent compound absorbs and emits photons in the ultraviolet band—between 400 and 10 nanometers—of the electromagnetic spectrum. The compound is chosen to avoid interference by UV radiation from the sun. The Earth's atmosphere absorbs as much as 99% of the UV radiation emitted by the sun in the 150-320 nm range. Thus the most advantageous luminescent compound absorbs and emits photons with wavelengths below 320 nm.

As an alternative to luminescent compounds that absorb and emit photons in the 150-320 nm range, compounds that absorb and emit photons of wavelengths greater than 320 nm can be used under certain circumstances. For example, these compounds could be used during nighttime conditions or in an enclosed UV-blocking environment such as a windowless structure.

The luminescent compound can be incorporated into the nucleic acid or the support platform in a number of different ways. For example, the compound can be entirely separate from the nucleic acid or the support platform. The compound can form a layer on the exterior surface of the nucleic acid or the support platform. The compound could also coat the interior surface of the encapsulant, or be incorporated into the encapsulant. In several of the described embodiments, the encapsulant layer must be designed to prevent inhibition of excitation and emission wavelengths.

If the seeded nucleic acid or support platform contains a photoluminescent compound, electromagnetic waves can be used to detect the presence of the tags at a distance. Scanning equipment shines photons of the excitatory wavelength on the object of interest and looks for photons emitted at the proper wavelength as determined by the compound used in the tags. Detection of photons with the correct wavelength suggests that a nucleic acid-labeled tag is present and alerts the scanner that further investigation is necessary. The advantage of this system is that the scanning equipment and tag can be designed such that the individual doing the scanning does not have to be in close proximity to the object of interest.

The detection process can be automated. An individual or object of interest can be forced to travel through a scanning point containing excitation equipment and emission detection equipment. As the individual or object of interest travels through the scanning point, the equipment scans for emitted photons of a certain wavelength. When the emitted photons are detected, a computer at the scanning point automatically alerts a remotely-located entity that subsequent analysis is necessary.

In yet another embodiment of the current invention, the detected nucleic acids taken from the exterior of an object are analyzed using any method that determines the exact order of nucleotide bases. There are currently a number of different commonly-used sequencing techniques including but not limited to dye-terminator sequencing, parallel sequencing, and sequencing by ligation. Sequencing machines allow automated sequencing and can be run 24 hours a day. If PCR techniques are used, the appropriate primers are chosen based upon the types of nucleic acid and/or tags known to be in the location of interest. Prior to sequencing or amplification, it is necessary to dissolve or otherwise remove an encapsulant layer from the tag in a manner that avoids inhibition of the downstream sequencing or PCR reactions, if such a layer is present or suspected to be present. In the preferred embodiment, the encapsulant and/or agglomerate is disrupted by bead beater, a form of mechanical disruption. This one-step method avoids chemicals or extractions which could affect or inhibit PCR reactions.

In addition to the traditional sequencing techniques described above, real-time PCR and sequencing by hybridization techniques allow rapid detection of target nucleic acids. According to the real-time PCR technique, the extracted nucleic acid is placed into a well or tube that has been pre-loaded with all reagents necessary for a PCR reaction as well as a sequence-specific, nucleotide-based, fluorescently-labeled probe. As the extracted nucleic acid is amplified, the polymerase degrades the probe and releases the fluorescent reporter. The reporter immediately fluoresces and alerts the system to the presence of a nucleotide. Under the sequencing by hybridization technique, the extracted nucleic acid is labeled with a fluorescent marker and is hybridized to a DNA microarray that contains the complementary nucleotide sequence from known seeded nucleic acid. If the extracted nucleic acid hybridizes to any of the complementary nucleic acid, the fluorescent signal alerts the system to the presence of a target nucleic acid. Since both methods of analysis avoid additional analysis and require relatively inexpensive analytical equipment, they promote faster and more affordable generation of data.

There are many other methods of detection of the nucleic acid and/or nucleic acid tag. For example, the nucleic acid can be detected using any molecular technique known to be suitable or adaptable for nucleic acid quantification or qualification, including but not limited to qPCR, high resolution melt (“HRM”), mass spectrometry, direct sequencing, strand displacement, and microarrays, among many others.

To characterize identified nucleic acid, the sequences obtained from the identified nucleic acid are compared to a database of sequences attached to seeded nucleic acid at step 250 of the method depicted in FIG. 2. To efficiently determine the point of origin or recent travel history of an object, individuals analyzing nucleic acid detected in the field will need access or information about the nucleic acid dispersed by the seeders. A database of seeded nucleic acid will require maximum security measures to avoid improper access and manipulation, including access protection measures such as passwords. Standard computer algorithms are used to find exact or approximate matches between a sequence in the field and a tag sequence in the database. Once such a match is found, the user can reasonably suspect that the object of interest has recently traveled through the location seeded by that nucleic acid. If the real-time PCR or sequencing by hybridization techniques are used, the identification of the seeded nucleic acid is quickly determined by equipment that scans the plate or microarray for fluorescent label.

Step 260 of FIG. 2 is an optional step which is only required if the user is attempting to backtrack the route taken by an object of interest or extrapolate the object's point of origin. According to some uses of the present invention, simply learning that a person or object has traveled through a particular location is sufficient information. For other uses, it is necessary to analyze the sequences of multiple tags. To extrapolate a route taken or a point of origin, the seeded tag location information obtained by analyzing the surfaces of the object is fed into a computer algorithm that quickly plots every potential route that the object has traveled based upon the possible combinations of tag locations. A similar algorithm can be used to extrapolate a point of origin based upon the identified tag locations.

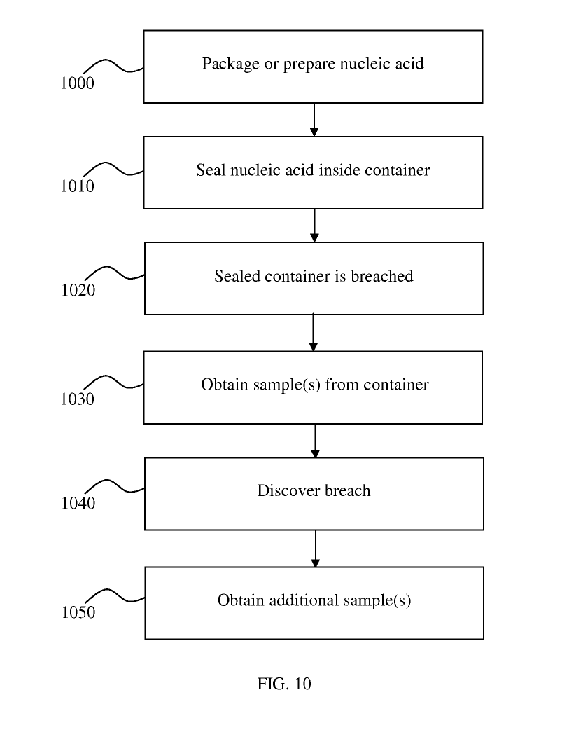

In another application of the nucleic acid tag, the tag is used to detect tampering. The flowchart in FIG. 10 summarizes a method of detecting tampering in accordance with one embodiment. At step 1000 in the method depicted in FIG. 10, the nucleic acid is packaged, prepared, or otherwise modified using any of a wide variety of methods, including but not limited to the methods described above. At step 1010, the prepared nucleic acid is sealed inside the item or object of interest. In a preferred embodiment, the nucleic acid is placed inside or on or in the interior of the container, item, or object of interest. The nucleic acid could simply be placed there prior to the item being sealed or closed, or a more complicated form of inserting, planting, or seeding the nucleic acid could be used. The nucleic acid can be placed or seeded by hand, or can be placed or seeded using mechanics or an automated process, or a combination of methods can be used.

Indeed, novel ways release nucleic acid into a container, item, or object of interest would be beneficial. According to one embodiment, the nucleic acid is disseminated into an object or item of interest using a microcontroller connected to a light sensor and an electronic match. The nucleic acid would be sealed into a small vessel that contains a minute amount of explosives. The microcontroller would be programmed to ignite the match when the light sensor indicates that the container is closed. The microcontroller would have an integrated timing circuit to prevent accidental tag release. When the match ignites, the explosives would react, forcing the nucleic acid out of the containment vessel and into the sealed container. Many other methods of introducing nucleic acid into a container, item, or object of interest can be used.

As another example of seeding an object of interest with the prepared or packaged nucleic acid, the nucleic acid can be associated with or incorporated into security components such as a security seal, tape, ink, or glue. For example, the nucleic acid tag can be placed between two layers of a security seal. When the seal is broken, the nucleic acid tag is released from between the layers of the seal, thereby indicating tampering. As another example, the nucleic acid tag can be associated with or incorporated into tape used to seal or shut an item of interest. When the tape is removed or altered, the nucleic acid tag is released, thereby indicating tampering. This system could be especially beneficial if the tampering individual is unaware of the nucleic acid tag's presence but attempts to replace the security seal, tape, or glue after tampering. Although the tampering may not be visually evident, it will be detected due to the release of the nucleic acid tag from the seal, tape, or glue.

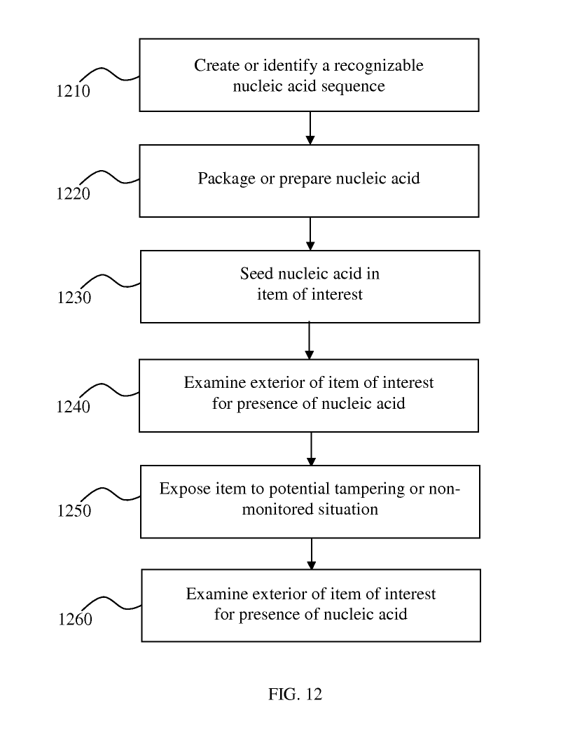

As an optional step, the exterior of the container can be sampled immediately or soon after the nucleic acid is sealed inside, as depicted in step 1260 of FIG. 12. This optional step confirms that the nucleic acid used for tamper detection was not inadvertently placed, or did not otherwise find its way, onto the exterior of the container, item, or object of interest. Nucleic acid located on the exterior of the container, item, or object of interest prior to deployment, storage, or use of the item will result in false positives when the object undergoes downstream analysis.

At step 1020 of the method, the sealed item of interest is breached, altered, tampered with, or otherwise modified in such a way as to release some of the nucleic acid sealed inside the item of interest. For example, if the item of interest is a container of goods, the nucleic acid sealed inside the container could be released if the container is opened or damaged. As just one example, medical goods such as pharmaceuticals are often shipped or distributed long distances, exposing them to potential tampering. It is vital, however, that the pharmaceuticals are not modified, altered, or tampered with during shipping or distribution. Accordingly, the packaging containing pharmaceuticals can be sealed with the prepared nucleic acid inside. If the packaging is tampered with, nucleic acid will be released and tampering can be detected.

At step 1030 of the method, one or more samples are obtained from the exterior of the item of interest in order to determine whether the sealed nucleic acid has been released, and thus whether there has been tampering. The sample can be analyzed using any method capable of: (i) detecting nucleic acid or the platform; and, optionally, (ii) determining the order of the nucleotide bases in the nucleic acid (in order to obtain any information stored within). PCR amplification and SNP genotyping are just two examples of methods that can detect the nucleic acid and determine a sequence of or within that nucleic acid.

At step 1040 of the method, analysis of the sample(s) taken from the item of interest reveals that there is nucleic acid present, and thus that the item has been damaged, tampered with, or otherwise modified. Further investigation will be required to determine when or how the item was modified, and who performed the modification. For example, at optional step 1050 of the method depicted in FIG. 10, further samples can be obtained in order to examine questions related to the tampering. Handlers may be sampled to determine if they have been labeled with the nucleic acid. Other surfaces, including locations through which the item traveled, can also be sampled to analyze the tampering. If the item traveled through multiple locations such as a truck, a warehouse, and a distribution center, each of these locations can be sampled to, for example, learn more about when and where the tampering occurred, and to create an approximate timeline of the item and the tampering.

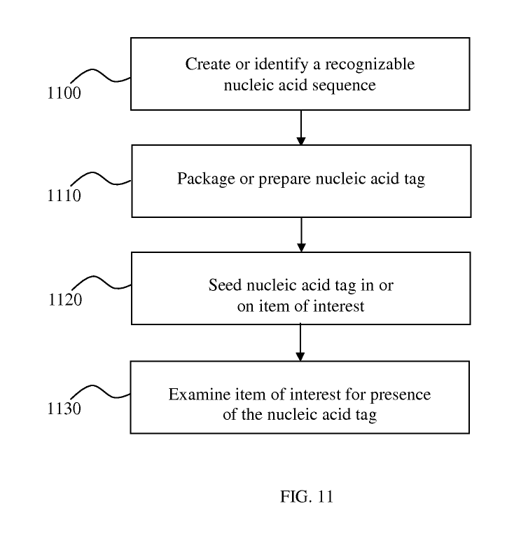

In addition to directly detecting tampering, the nucleic acid tags described herein can be used for authenticating an object or thing. FIG. 11 is a schematic representation of an embodiment of an authentication method according to one aspect of the invention. More specifically, the figure represents a method for authenticating an object that has been labeled with a seeded nucleic acid tag. The item can be, for example, any person or object of interest.

As an initial step 1100 of the method, a suitable nucleic acid sequence is characterized or created according to any of the methods described herein. In one embodiment, the sequence ranges from a short oligonucleotide to an entire genome and is generated through any of the various known methods of natural or artificial nucleic acid synthesis. The nucleic acid can be completely composed of either natural nucleic acids which normally compose the genomes of organisms, artificial nucleic acids, or any combination thereof. In another embodiment, the nucleic acid molecules contain primer-binding sequences surrounding unique nucleotide sequences. The unique nucleotide sequence contained between the primers can encode information that corresponds to an identification, location, date, time, or other data specific to that unique sequence. Since analysis of every nucleic acid molecule can use the same primers, the analysis can be performed faster and more efficiently.

The nucleic acid tag can be used not only for simple binary (i.e., “yes/no”) authentication, but also for informational authentication. In the example of a pharmaceutical label, the nucleic acid tag can not only verify that the item is authentic, it can further comprise information about the pharmaceutical's components, date of manufacture, date of expiration, place of manufacture, lot number, and many other pieces of information. In the example of a food label, the nucleic acid tag can not only verify that the item is authentic, it can further comprise information about the food's components, the location it was grown and/or processed, date of processing, date of expiration, the lot number, and many other pieces of information.

At step 1110 of the method shown in FIG. 11, the nucleic acid is packaged, prepared, or otherwise modified prior to use. Preparation of the nucleic acid can range from little or no preparation or modification to an extensive series of steps for modifying the nucleic acid. For example, the nucleic acid can be used to derivatize nanoparticles, as described herein, or can be added to another structure or base. As another example, the nucleic acid can be packaged into an appropriate tag complex as described elsewhere in this specification.

At step 1120 of the method depicted in FIG. 11, an item or object of interest to be authenticated is seeded with the prepared or packaged nucleic acid. The nucleic acid tag can be placed inside or on or in the interior of the container, item, or object of interest. The nucleic acid could simply be placed there prior to the item being sealed or closed, or a more complicated form of inserting, planting, or seeding the nucleic acid could be used. The nucleic acid can be placed or seeded by hand, or can be placed or seeded using mechanics or an automated process, or a combination of methods can be used. For example, the nucleic acid tag can be associated with or incorporated into security components such as a security seal, tape, ink, or glue. The nucleic acid tag can be placed between two layers of a security seal, or can be associated with or incorporated into tape used to seal or shut an item of interest.

As yet another example, the nucleic acid tag can be seeded or placed in or on or otherwise associated with a sensitive product. The nucleic acid tag can be associated with or otherwise seeded in or on a label of a pharmaceutical, food, medicine, or other commercially or security sensitive object.

As another example, the tag can be incorporated into the material comprising all or a portion of the actual item or object of interest. For example, the tag can be seeded into credit cards, or another plastic or polymer structure, by seeding the tag directly into a precursor component such as the PVC, PVC-Co-A, or other polymer precursor before the credit card is formed. Given the ubiquitous nature of plastic and complex polymers in all aspects of manufacturing, distribution, and storage, for example, there are an almost unlimited number of possible applications for this seeding technique.

Once the item of interest to be authenticated is labeled or otherwise seeded with the nucleic acid tag, the container, item, or object of interest is allowed to be used for the purpose for which the tag was designed. In other words, the object can be exposed to situations where authentication may be necessary. For example, the object can be shipped, deployed, moved, stored, or otherwise used, among many other options. During any of these steps or uses, the object of interest can be exposed to situations where it may be illicitly tampered with. In addition to detecting illicit access or other tampering or alteration of an object, the seeded nucleic acid can be used to detect breakage, leakage, damage, severe movement, or many other types of motion or activity that an object of interest may be exposed to during routine or specialized functioning.

At step 1130 of the method depicted in FIG. 11, the authenticity of the container, item, or object of interest can be confirmed by determining the presence of seeded nucleic acid using any of the methods described herein. For example, once an object of interest is identified, the object can be examined for seeded nucleic acid using any mechanism designed to pick up nucleic acid from the surfaces of the object. For example, the exterior of the object of interest can be swabbed for nucleic acid and/or tags. The nucleic acid can be identified and characterized using any of the methods, systems, devices, or molecular techniques described or mentioned herein.

Although the present invention has been described in connection with a preferred embodiment, it should be understood that modifications, alterations, and additions can be made to the invention without departing from the scope of the invention as defined by the claims.

What is claimed is:

1. A nucleic acid tag comprising:
an agglomerated plurality of nucleotide-support platforms each attached to a plurality of nucleic acid molecules, each of said nucleic acid molecules comprising identifying information, wherein a spacer is located between said nucleotide-support platform and said identifying information, and further wherein each of said plurality of nucleotide-support platforms comprises a diatom having a diameter of approximately 1 micrometer or greater; and
an encapsulant surrounding the agglomerated plurality of nanoparticle nucleotide-support platforms and said plurality of nucleic acid molecules.

2. The nucleic acid tag of claim 1, wherein the encapsulant is adapted to prevent degradation of the plurality of nucleic acid molecules.

3. The nucleic acid tag of claim 1, wherein each of the plurality of nucleic acid molecules is composed of nucleotides selected from the group consisting of ribonucleotides, deoxyribonucleotides, and nucleotide analogues.

4. The nucleic acid tag of claim 1, wherein each of the plurality of nucleic acid molecules is an oligonucleotide.

5. The nucleic acid tag of claim 1, wherein each of the plurality of nucleic acid molecules is genomic deoxyribonucleic acid ranging from two nucleotides to the entire genome.

6. The nucleic acid tag of claim 1, wherein information is encrypted within the genomic deoxyribonucleic acid molecule by altering the sequence of nucleotides.

7. The nucleic acid tag of claim 1, wherein the nucleic acid tag comprises a retroreflector.

8. The nucleic acid tag of claim 1, wherein the nucleic acid tag comprises a luminescent compound.

9. A method for determining whether an item has moved through a geographic location, the method comprising:
creating a nucleic acid tag comprising a nanoparticle nucleotide-support platform attached to a plurality of nucleic acid molecules, each of said nucleic acid molecules comprising identifying information, wherein a spacer is located between said nanoparticle nucleotide-support platform and said identifying information, and further wherein said nanoparticle nucleotide-support platform comprises diatomaceous earth;
seeding the geographic location with the nucleic acid tag; and
examining the item for the presence of the nucleic acid tag.

10. The method according to claim 9, wherein the nucleic acid tag is analyzed by sequencing all or part of the nucleic acid molecule.

11. The method according to claim 9, wherein each geographic location is seeded with a unique nucleic acid tag.

12. A method for backtracking the travel history of an item, the method comprising:
creating two or more nucleic acid tags, each tag comprising: a nanoparticle nucleotide-support platform attached to a plurality of nucleic acid molecules, each of said nucleic acid molecules comprising identifying information, wherein a spacer is located between said nanoparticle nucleotide-support platform and said identifying information, and further wherein said nanoparticle nucleotide-support platform comprises diatomaceous earth;
seeding each of two or more geographic locations with said nucleic acid tags, wherein each geographic location is seeded with a unique nucleic acid;
examining said item for the presence of one or more nucleic acid tags; and
identifying the geographic location associated with each nucleic acid tag detected on said item.

13. The method for determining the point of origin of an item according to claim 12, the method further comprising:
extrapolating the point of origin.

14. A method for detecting a seeded nucleic acid tag in or on an item of interest, the method comprising:
obtaining a nucleic acid tag, wherein said nucleic acid tag comprises a nanoparticle nucleotide-support platform attached to a plurality of nucleic acid molecules, each of said nucleic acid molecules comprising identifying information, wherein a spacer is located between said nanoparticle nucleotide-support platform and said identifying information, and further wherein said nanoparticle nucleotide-support platform comprises diatomaceous earth;
adding the nucleic acid tag to the item of interest;
sampling a portion of the item of interest for the presence of the nucleic acid tag; and
detecting the presence of the nucleic acid tag in the sample.

15. The method of claim 14, wherein the presence of the tag on an exterior surface indicates tampering.

16. The method of claim 14, wherein the presence of the nucleic acid tag authenticates the item of interest.

17. The method of claim 14, wherein the step of adding the nucleic acid tag to the item of interest comprises incorporating the nucleic acid tag within a label or a package of the item of interest.

18. The method of claim 14, wherein the step of adding the nucleic acid tag to the item of interest comprises incorporating the nucleic acid tag into a precursor of the item of interest.

19. The method of claim 14, wherein the plurality of nucleic acid molecules are synthesized directly onto the nanoparticle nucleotide-support platform.
